(19) United States
(12) Patent Application Publication
(10) Pub. No.: US 2021/0307616 A1
(43) Pub. Date: Oct. 7, 2021

(54) Fever Detector

(71) Applicant: Marshall STEWMAN, Woodway, TX (US)

(72) Inventor: Marshall STEWMAN, Woodway, TX (US)

(21) Appl. No.: 17/037,373

(22) Filed: Sep. 29, 2020

Related U.S. Application Data
(60) Provisional application No. 63/003,352, filed on Apr. 1, 2020.

(51) Int. Cl. A61B 5/01; G01J 5/00; G01J 5/10; G01J 5/02; A61B 5/00

(52) U.S. Cl. CPC A61B 5/01; G01J 5/0025; G01J 5/10; A61B 2560/0431; G01J 5/0265; A61B 5/742; G01J 5/0205

(57) ABSTRACT

A system for monitoring temperature of an individual. The system includes a support housing with at least one temperature sensor. The temperature sensor is coupled to at least one temperature output. Individuals can walk adjacent to or through the support and have their temperature taken. Additionally, the support can be toward and around individuals.

### PRIORITY

The present invention claims priority to U.S. Provisional Application No. 63/003,352 filed Apr. 1, 2020, the entirety of which is hereby incorporated by reference.

### BACKGROUND OF THE INVENTION

### Technical Field

The present invention relates to a system and method for monitoring temperatures.

### Description of Related Art

The world is more connected today than anytime in history. Consequently, diseases and bacterial can spread at an alarming rate. A virus can originate in a small town and spread across the globe in a month. Often tests to determine if a person is carrying the virus or disease is not widely available. Consequently, there is a need to monitor and assess symptoms of a disease with a large volume of people.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 is a schematic of a monitoring system in one embodiment;

FIG. 2 is a view of the outputs in one embodiment;

FIG. 3 is a side view of an aimable sensor one embodiment.

### DETAILED DESCRIPTION

Several embodiments of Applicant's invention will now be described with reference to the drawings. Unless otherwise noted, like elements will be identified by identical numbers throughout all figures. The invention illustratively disclosed herein suitably may be practiced in the absence of any element which is not specifically disclosed herein.

As noted, often tests to determine if a person is infected with a disease or has been exposed to a virus is not widely available. Often people are concentrated or congregated in large venues where it is desirable to separate exposed persons from unexposed persons. Even when a determinative test is not available, often the symptoms of the disease or virus can be monitored to determine if a person has been exposed. A virus, such as a flu, may result in many different symptoms. One such symptom is a fever, or increased temperature. While not determinative of whether a person is positive for a specific virus, a fever can indicate that a person might carry the virus. That person can then be quarantined from the community to minimize spread.

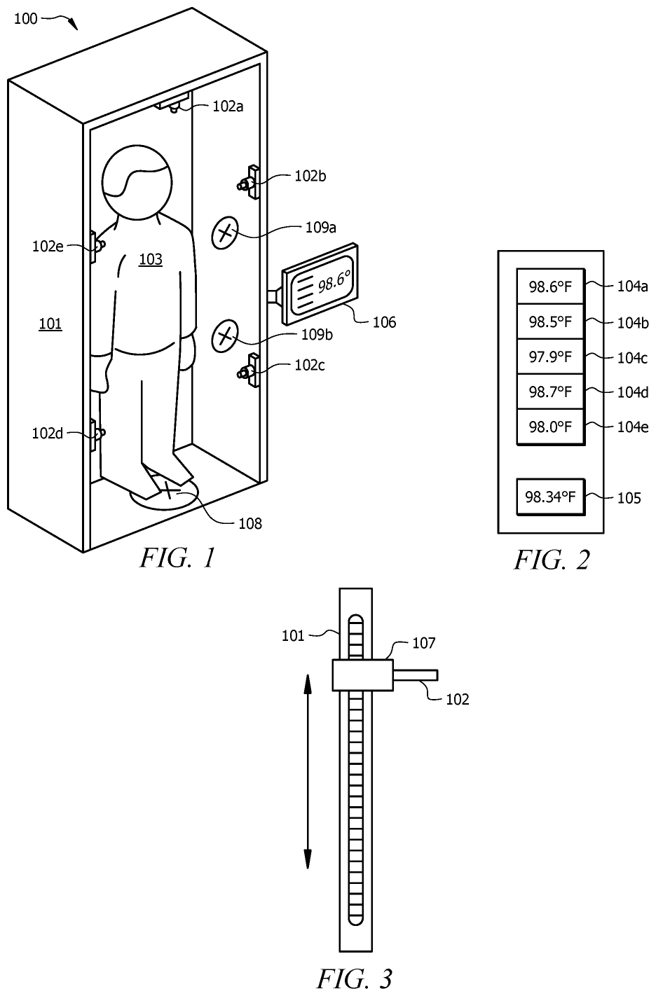

FIG. 1 is a schematic of a monitoring system in one embodiment. FIG. 1 shows a system utilizing a support 101 to house one or a plurality of temperature sensors 102. While a support 101 is demonstrated, this is for illustrative purposes only and should not be deemed limiting. A support 101 is a structure which can house or hold one or more temperature sensors 102. A support 101 can include a wall, door frame, or any other structure which can support a sensor.

In one embodiment, and as depicted, the support 101 comprises an external frame through which a user can walk. The support 101 can be similar to traditional metal detectors. The benefit of a support 101 such as a metal detector is that users are familiar with their use and understand to slowly walk through the support 101.

The support can comprise virtually any material. It can comprise plastic, wood, metal, rubber, and combinations thereof. The support 101 can be a standalone structure, or it can link or couple with a separate structure.

As noted, coupled to the support 101 is at least one temperature sensor 102. The temperature sensor 102 can comprise virtually any sensor which detects and determines a temperature. In one embodiment the temperature sensor 102 comprises an infrared temperature sensor. An infrared temperature sensor uses an infrared signal to determine the sensor of an object. Such sensors are accurate and very fast. The infrared sensor can be a stand alone sensor or it can be incorporated into other equipment such as cameras, iPads, etc.

While one embodiment utilizing an infrared temperature sensor 102, this is for illustrative purposes only and should not be deemed limiting. Virtually any temperature sensor 102 which is both accurate and fast can be utilized.

In one embodiment, and as depicted, the temperature sensor 102 determines and reads the individuals temperature without contacting the individual directly. This is beneficial because it increases speed and reduces transmission of diseases and virus. In other embodiments, however, the temperature sensor 102 may make contact with the tested individual.

In one embodiment the system 100 comprises one temperature sensor 102. In other embodiments, however, the system 100 comprises a plurality of temperature sensors 102. As depicted there are five temperature sensors 102a-e spaced along the inner circumference of the support 101.

Having a plurality of temperature sensors 102 in some embodiments offers superior results to a single temperature sensor. There are several reasons for this. First, having multiple temperature sensors increases the likelihood that a clear reading of the user can be obtained. For example, the sensor often needs to read a user's skin to obtain an accurate result. Reading a person's coat or external clothing will not always provide accurate results. Consequently, having multiple sensors increases the likelihood that a person's skin can be read.

The location of the sensors 102 can be modified and/or adjusted to increase accurate readings. For example, while the sensors 102 are spaced along the interior periphery, in other embodiments the sensors 102 will be targeted to read a user's face.

In one embodiment the sensors 102 are stationary. In another embodiment the sensors 102 are stationary but adjustable. For example, the sensors 102 can be adjusted laterally to be positioned for height. In one such embodiment there will be a lower sensor 102 for children and an elevated sensor for adults.

Second, having a plurality of readings offers an opportunity to review data from multiple sensors and apply analytics to this data. FIG. 2 is a view of the outputs in one embodiment. As shown, the temperature outputs 104a-e from the corresponding temperature sensors 102a-e are displayed. The analytic output 105 is displayed. The analytic output 105 can be a simple average of the read temperatures, or more sophisticated analytics can be applied. For example, the analytics can apply standards to remove clearly erroneous readings. If, for example, one temperature sensor 102 reports a result of 135° F., then the person clearly did not have a fever of 135° F. Rather, it is likely that the user's coat button had been left in a hot car. If this data point is included in the analytics, a false positive of a fever would result. Likewise, if a reading of 40° F. is reported, this was likely reading the user's cold drink rather than the temperature of the user. If this lower data point was utilized, the resulting number may not be properly indicative of the user's actual temperature. Consequently, in one embodiment the system has analytic abilities to filter data to ensure accuracy.

The temperature outputs 104 and analytic output 105 can be stored and/or displayed in any available medium, including but not limited to, a computer, a laptop, a smart phone, a tablet, etc. Further, the data may be sent to a remote server for processing and only the analytic output 105 is sent locally to be viewed.

Furthermore, aside from only collecting temperature information, in other embodiments the system stores identification information. Identification information is any information which relates and pertains to the user. Identification information can be biometric data such as a fingerprint, retinal scan, etc. Identification information can also comprise scanning an ID badge, or even manually entering a person's name. The identification information can be used with contact tracing in the event a user tests positive for a specific virus, disease, etc.

In other embodiments the system further comprises a counter. A counter can be a separate physical counter which counts the users which pass through the support 101. This can include a light sensor which detects and records movement. In other embodiments, however, the counter is analytical in that it records the number of readings taken. As an example, if ten people walk through the support 101, the physical counter would count ten people. Likewise, the system would have ten separate results and therefore infer that ten people walked through the support 101. Being able to count individuals is a benefit for a variety of reasons. First, a space such as a restaurant or concert venue may only allow so many people to attend. The counting ability provides proof that the venue, restaurant, etc. is in compliance. Second, counting the number of users provides helpful statistical data. The system can say, for example, that only 2% of the users exhibited a fever. That can be useful in studies as well as contract tracing.

Turning to FIG. 1, in one embodiment the system comprises at least one alignment indicator. As depicted, there is a horizontal alignment indicator 108 and a vertical alignment indicator 109a, b. The horizontal alignment indicator 108 notifies the user where to stand. The horizontal alignment indicator 108 can comprise a marking, such as an “X” or other indicator of where the user should stand while getting their temperature read. The horizontal alignment indicator 108, in some embodiment, aids the sensor 102 in that it ensures the user is a desirable distance and location for a proper reading.

Likewise, in one embodiment, and as depicted, the system can comprise vertical alignment indicators 109a, b. As shown, there is a taller alignment indicator 109a for taller individuals and a comparatively shorter indicator 109b for shorter individuals. This encourages individuals to face a specific direction to aid the sensor 102 in obtaining a correct and unobstructed view. The location and placement of the alignment indicators can vary depending upon application.

In some embodiments the user simply walks through or adjacent to the support structure 101 to get their temperatures taken. The user need not move or stop, but can simply walk through without breaking stride. In other embodiments, however, the user must stop at the alignment indicators and place their head, or at least look in a specified location to get the temperature read. In other embodiments discussed below, however, the sensors 102 are not stationary.

Turning back to FIG. 1, the sensors 102 can be stationary, or the sensors 102 can move. As but one example, in one embodiment there are a plurality of stationary sensors 102 positioned to read a user's forehead. In another embodiment, however, the sensor 102 is moveable laterally to adjust to a person's height. If a person is six and a half feet tall, the system will recognize this and raise the sensor 102 to read the person's forehead. If the next user is a 3-year-old, the sensor 102 will lower to obtain the correct height.

In still another embodiment the sensors 102 can pivot up or down, and/or left and right. Put differently, the sensors 102 can be aimed. FIG. 3 is an embodiment with an aimable sensor. As shown the sensor 102 is attached to a moveable carriage 107. The carriage 107 can move up or down along the length of the support depicted 101. In other embodiments, the carriage 107 can also move left and right (in and out of the page) along the support 101.

The sensor 102 can be affixed to a fixed point, but the sensor 102 is aimed toward the user's forehead depending on the user's height and location. In such embodiments the system comprises other sensors which read a user's height and location and provides the temperature sensor 102 the necessary information to obtain a proper temperature measurement.

In still other embodiments the sensor 102 is both moveable and aimable, as shown in FIG. 3. This increases the likelihood that a correct temperature will be recorded.

As noted above, even in embodiments wherein sensors 102 are moveable and/or aimable, it is often beneficial to have more than one sensor. For the reasons stated above, having more than one temperature sensors 102 increases the data available to be analyzed and increases the accuracy of the results. Additionally, having multiple sensors 102 can increase the throughput through the monitoring system 100. Rather than depending upon a single sensor 102 to obtain a result, if multiple sensors 102 are utilized, the likelihood of obtaining an accurate reading is increased.

FIG. 1 also depicts a result output 106. The result output 106 is an indicator of the results. As but one example, the result output 106 can show a green light if the user has no fever, or a red light if a fever has been detected. The result output 106 can also display the numerical results. In one embodiment the output 106 displays the numbers and the color results, whereas in other embodiments the output 106 just displays either a number or a color result. In still other embodiments the output 106 displays text such as “Enter” or “Do not enter.” Virtually any text, code, scheme, or number which displays whether the user has a fever or not can be utilized.

The result output 106 can be attached or coupled to the support 101 as depicted. In this result, an operator will monitor the result output 106 as individuals pass through the support 101. If the result output 106 indicates that a specific individual has a fever, that person can be asked to walk through the support 101 again to verify the output 106, or the person can be notified to quarantine. The result output 106 can comprise an electronic sign, display, tablet, screen etc. which displays results. In such embodiments the user can see the results. In other embodiments the result output 106 is sent remotely to an operator, and/or user's handheld device, smart phone, tablet, etc.

As noted, a monitoring system comprising a support 101, at least one sensor 102, a temperature output 104, and a result output 106 has been described. Now a method of utilizing the system will be described.

First, the monitoring system is powered. The temperature sensors 102 are calibrated as necessary. Thereafter, an individual is placed in proximity of at least one temperature sensor 102. The individual can walk through the support 101 as depicted in FIG. 1. In other embodiments, however, the individual simply walks or stands in a designated location adjacent to a temperature sensor. Thereafter, the sensors 102 read and interpret the individual's temperature. In embodiment utilizing multiple sensors 102, the data can be analyzed to remove obviously incorrect data points. The resulting analytic output 105 is then reviewed to determine if the individual has a fever. The result is depicted in a result output 106.

While a system has been described wherein the individual walks and moves relative to a stationary support 101, this is for illustrative purposes only and should not be deemed limiting. In other embodiments, for example, the monitoring system 100 moves relative to stationary individuals. Consider, for example, a long line of individuals. Rather than the individuals passing adjacent or through the support 101, the support 101 can move adjacent to stationary individuals. In this manner, the monitoring system 100 can move through a line or crowd of people and analyze individuals in the line. The support 101 can have wheels, tracks, or other methods of maintaining balance and providing propulsion to move the support 101 to and around individuals.

The system and method can be utilized in a variety of applications. In one embodiment the system can be placed in the entrance or exit of a location. For example, the system and method can be placed in the airport, outside churches, schools, restaurants, etc. The system can be used to scan individuals before allowing them to enter a specific location. The system can be used at customs to scan individuals before entering or exiting a country.

The system and method can also be placed in medical facilities such as hospitals or nursing homes. Often these facilities house individuals who are high risk for being impacted by viruses such as those with compromised immunity. The system can prevent an individual with a fever, known and unknown, from entering a nursing home. This results in containing the virus or disease and preventing further community transmission.

Additionally, as noted, fever is often a precursor or symptom of a virus or disease. In cases of limited tests, the system and method can be utilized to screen which individuals should be tested. This results in conserving of tests and only using the tests on individuals exhibiting systems.

The system and method discussed above can be inexpensive. As noted, the support 101 can comprise a frame structure with one or more temperature sensors 102. The speed of these temperature sensors 102 allow fast and efficient scanning and reading of the tested individuals. In other embodiments, as described above, the system can comprise more complicated and complex moving parts such as laterally and transversely moveable and aimable temperature sensors 102.

While the invention has been particularly shown and described with reference to a preferred embodiment, it will be understood by those skilled in the art that various changes in form and detail may be made therein without departing from the spirit and scope of the invention.

### Additional Description

The following clauses are offered as further description of the disclosed invention.

Clauses 1. A system for monitoring temperature of an individual, said system comprising:
a support housing at least one temperature sensor;
a temperature output coupled to said at least one temperature sensor.
   Clauses 2. The system of claim 1 further comprising a result output displaying results from said temperature sensor.
   Clauses 3. The system of claim 1 wherein said support comprises a void through which a user may walk.
   Clauses 4. The system of claim 1 comprising a plurality of temperature sensors.
   Clauses 5. The system of claim 1 further comprising a carriage coupled to said support.
   Clauses 6. The system of claim 5 wherein said carriage is moveable relative to said support, and wherein at least one temperature sensor is located on said carriage.
   Clauses 7. The system of claim 1 wherein said temperature sensor is aimable.
   Clauses 8. The system of claim 1 further comprising at least one horizontal alignment indicator.
   Clauses 9. The system of claim 1 further comprising at least one vertical alignment indicator.
   Clauses 10. The system of claim 1 further comprising an input for receiving identification information.
   Clauses 11. The system of claim 1 wherein said system further comprises a physical counter.
   Clauses 12. The system of claim 1 wherein said sensors are adjustable along said support.
   Clauses 13. The system of claim 1 wherein said at least one sensor comprises at least five sensors located along an inner circumference of said support.
   Clauses 14. The system of claim 1 wherein said support is stationary.
   Clauses 15. The system of claim 1 wherein said support is moveable on the ground.

What is claimed is:

1. A system for monitoring temperature of an individual, said system comprising:
a support housing at least one temperature sensor;
a temperature output coupled to said at least one temperature sensor.

2. The system of claim 1 further comprising a result output displaying results from said temperature sensor.

3. The system of claim 1 wherein said support comprises a void through which a user may walk.

4. The system of claim 1 comprising a plurality of temperature sensors.

5. The system of claim 1 further comprising a carriage coupled to said support.

6. The system of claim 5 wherein said carriage is moveable relative to said support, and wherein at least one temperature sensor is located on said carriage.

7. The system of claim 1 wherein said temperature sensor is aimable.

8. The system of claim 1 further comprising at least one horizontal alignment indicator.

9. The system of claim 1 further comprising at least one vertical alignment indicator.

10. The system of claim 1 further comprising an input for receiving identification information.

11. The system of claim 1 wherein said system further comprises a physical counter.

12. The system of claim 1 wherein said sensors are adjustable along said support.

13. The system of claim 1 wherein said at least one sensor comprises at least five sensors located along an inner circumference of said support.

14. The system of claim 1 wherein said support is stationary.

15. The system of claim 1 wherein said support is moveable on the ground.
